(19) United States
(12) Patent Application Publication
(10) Pub. No.: US 2024/0153629 A1
(43) Pub. Date: May 9, 2024

(54) METHOD AND SYSTEM FOR GENERATING A GRAPH NEURAL NETWORK COMPRISING ASSOCIATION OF ONE OR MORE ORGANS WITH A DISEASE

(71) Applicant: Innoplexus AG, Eschborn (DE)

(72) Inventors: Amit Agarwal, Pune (IN); Manojkumar Avghade, Pune (IN); Ishita Mallick, Pune (IN); Om Sharma, Pune (IN)

(73) Assignee: Innoplexus AG, Eschborn (DE)

(21) Appl. No.: 17/981,954

(22) Filed: Nov. 7, 2022

(51) Int. Cl. G16H 50/20; G16H 70/60

(52) U.S. Cl. CPC G16H 50/20; G16H 70/60

(57) ABSTRACT

A method and system for generating a graph neural network comprising association of one or more organs with a disease. The method enables target identification of organs to expediate the drug purposing or discovery process. The method and system is an implementation of digital phama to accelerate target identification for drug design. The method comprises identifying one or more organs associated with a disease from one or more categories, wherein the one or more categories comprises of an organ ontology, one or more information indicative of the disease, plurality of abstract of publications. Weights are assigned to each of the one or more categories and the identified one or more organs, wherein the weights are assigned to the identified one or more organs based on one or more of—frequency of the keywords appearing the one or more categories, and one or more location of the keywords appearing the plurality of abstracts of publications. The method further comprises normalizing and summing the assigned weights to determine a probability and association indicative of the one or more organ being associated with the disease to generate a graph neural network of the one or more organs associated with the disease. The graph neural network is based on the probability and the association indicative of the one or more organ being associated with the disease.

### FIELD OF TECHNOLOGY

Certain embodiments of the disclosure relate to associating one or more organs with a disease. More specifically, certain embodiments of the disclosure relate to method and system for generating a graph neural network comprising association of one or more organs with a disease.

### BACKGROUND

In the drug discovery process, target identification and characterization begin with identifying the function of a possible therapeutic target (gene/protein) and its role in the disease. Target protein expression is one of the important target characteristics which could be utilized for prioritization of the targets.

The main organ/tissue affected in an indication is most likely to be involved in the pathophysiology of the disease. Identification of this organ/tissue based on the indication name is not always possible. Further information on the description of the disease or the actual pathophysiology may be necessary in order to identify the organ/tissue.

Currently we have to manually identify the indication relevant organ by understanding the disease pathology and then input it to check for protein expression. This process is time-consuming and requires human intervention to identify the correct organ/tissue.

Further limitations and disadvantages of conventional and traditional approaches will become apparent to one of skill in the art, through comparison of such systems with some aspects of the present disclosure as set forth in the remainder of the present application with reference to the drawings.

### OBJECT OF THE INVENTION

The objective of the inventive is to association one or more organs with one or more disease to improve target identification it the early drug discovery process.

The objective of the invention is to generate a graph neural network comprising association of one or more organs with a disease.

Another objective of the invention is to identify the organ affected with the disease, wherein the identification includes a probability score.

Yet another objective of the invention is to identify primary and second organs affected with a disease.

Further objective of the invention is to identify organ-based hierarchy of diseases.

Another objective of the invention is to generate disease similarity score for one or more diseases based on the graph neural network for one or more organ associated with one or more disease to improve target identification in the drug discovery process.

Furthermore, another objective of the invention is to identify novel associations between diseases based on the generated network.

Yet another objective of the invention is to identify association of one or more organs with a disease from various static and dynamic sources, wherein the dynamic sources relate to latest publications comprising association between organ and disease. In an example, a hypothesis of the effects of COVID-19 in mental health could be a dynamic association between organ and disease. The static sources are the established disease definitions.

Further objective of the invention is to assign weights in consideration of the one or more categories and location of the association between the one or more organs and disease based on the importance of the categories and location in view of pathophysiology of the disease.

Furthermore, another objective of the invention is to determine a probability of the one or more organ being associated with the disease.

Furthermore, another objective of the invention is to determine association indicative of the one or more organ being associated with the disease.

Moreover, another objective of the invention is to generate the graph neural network on the varying probabilities and association indicative of the one or more organ being associated with the disease.

### BRIEF SUMMARY OF THE DISCLOSURE

A method is disclosed for generating a graph neural network comprising association of one or more organs with a disease, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram that illustrates an exemplary system for generating a graph neural network comprising association of one or more organs with a disease, in accordance with an exemplary embodiment of the disclosure.

FIGS. 2A, 2B and 2C depicts visual representation of the steps and outputs derived from the various implementations of generating a graph neural network comprising association of one or more organs with a disease, in accordance with an exemplary embodiment of the disclosure.

FIG. 3 depict flowcharts illustrating exemplary operations for generating a graph neural network comprising association of one or more organs with a disease, in accordance with various exemplary embodiments of the disclosure.

FIG. 4 illustrates an example of a CNN Model architecture for comparing the graph neural network of the one or more organs associated with one or more diseases, in accordance with an exemplary embodiment of the disclosure.

### DETAILED DESCRIPTION OF THE DISCLOSURE

Certain embodiments of the disclosure relate to generating a graph neural network comprising association of one or more organs with a disease. In the context of the current invention, the association of one or more organs with a disease is being computationally identified from a plurality of datasets using method claimed herein.

In accordance with various embodiments of the disclosure, a method is provided for generating a graph neural network comprising association of one or more organs with a disease. The method comprises identifying one or more organs associated with a disease from one or more categories, wherein the one or more categories comprises of an organ ontology, one or more information indicative of the disease, plurality of abstract of publications. The method further comprises assigning weights to each of the one or more categories and the identified one or more organs, wherein the weights are assigned to the identified one or more organs based on one or more of—frequency of the keywords appearing the one or more categories, and one or more location of the keywords appearing the plurality of abstracts of publications. The method further comprises normalizing and summing the assigned weights to determine a probability and association indicative of the one or more organ being associated with the disease. Furthermore, the method comprises generating a graph neural network of the one or more organs associated with the disease, wherein the graph neural network is based on the probability and the association indicative of the one or more organ being associated with the disease

In accordance with an embodiment, the method comprises generating disease similarity score for one or more diseases based on the graph neural network for one or more organ associated with one or more disease.

In accordance with an embodiment, the disease similarity score is generated based on implementing a CNN Model architecture for comparing the graph neural network of the one or more organs associated with one or more diseases.

In accordance with an embodiment, comprises creating, by one or more processors, the organ ontology, wherein the organ ontology comprises organs, tissues, cells and its associated keywords.

In accordance with an embodiment, creating the organ ontology comprises parsing plurality of organ datasets to create the organ ontology.

In accordance with an embodiment, the method comprises extracting information indicative of the disease from a plurality of disease datasets.

In accordance with an embodiment, the method comprises extracting plurality of abstract from a plurality of publication datasets.

In accordance with an embodiment, the one or more locations in the abstract of the publication comprising introduction, methods & materials, and conclusion.

In accordance with an embodiment, a higher weightage is assigned to the identified one or more organs for higher frequency of the keywords appearing in—the one or more information indicative of the disease, and methods & materials location of the one or more location in the abstract.

In accordance with an embodiment, the probability output denotes a probability of the said organ being associated with the respective disease, and the association indicative of the one or more organ being associated with the disease specifies a combination of the one or more organs extracted from each of the abstract of the publication.

In accordance with another aspect of the disclosure, a system generating a graph neural network comprising association of one or more organs with a disease. The system comprises an organ ontology, a plurality of disease datasets, a plurality of publication datasets, and at least one server. The at least one server is communicably coupled with the organ ontology, the plurality of disease datasets, and the plurality of publication datasets. The comprising one or more processors configured to identify one or more organs associated with a disease from one or more categories, wherein the one or more categories comprises of an organ ontology, one or more information indicative of the disease, plurality of abstract of publications, assign weights to each of the one or more categories and the identified one or more organs, wherein the weights are assigned to the identified one or more organs based on one or more of—frequency of the keywords appearing the one or more categories, and one or more location of the keywords appearing the plurality of abstracts of publications, normalize and sum the assigned weights to determine a probability and association indicative of the one or more organ being associated with the disease, and generate a graph neural network of the one or more organs associated with the disease, wherein the graph neural network is based on the probability and the association indicative of the one or more organ being associated with the disease.

In accordance with an embodiment, the at least one server is configured to generate disease similarity score for one or more diseases based on the graph neural network for one or more organ associated with one or more disease.

In accordance with an embodiment, the disease similarity score is generated based on implementing a CNN Model architecture for comparing the graph neural network of the one or more organs associated with one or more diseases.

In accordance with an embodiment, the organ ontology is created by parsing plurality of organ datasets.

In accordance with an embodiment, the one or more locations in the abstract of the publication comprising introduction, methods & materials, and conclusion

In accordance with an embodiment, a higher weightage is assigned to the identified one or more organs for higher frequency of the keywords appearing in—the one or more information indicative of the disease, and methods & materials location of the one or more location in the abstract.

In accordance with an embodiment, the probability output denotes a probability of the said organ being associated with the respective disease, and the association indicative of the one or more organ being associated with the disease specifies a combination of the one or more organs extracted from each of the abstract of the publication.

In accordance with another aspect of the disclosure, a computer program product comprising a computer useable medium having computer program logic recorded thereon for enabling a processor to generate a graph neural network comprising association of one or more organs with a disease. The computer program product comprising of a computer program logic identifying one or more organs associated with a disease from one or more categories, wherein the one or more categories comprises of an organ ontology, one or more information indicative of the disease, plurality of abstract of publications, assigning weights to each of the one or more categories and the identified one or more organs, wherein the weights are assigned to the identified one or more organs based on one or more of—frequency of the keywords appearing the one or more categories, and one or more location of the keywords appearing the plurality of abstracts of publications, normalizing and summing the assigned weights to determine a probability and association indicative of the one or more organ being associated with the disease, and generating a graph neural network of the one or more organs associated with the disease, wherein the graph neural network is based on the probability and the association indicative of the one or more organ being associated with the disease.

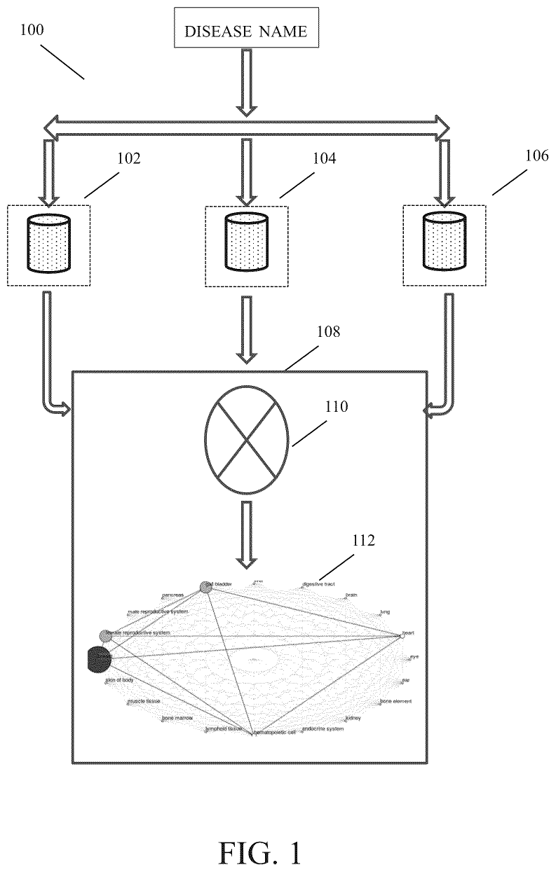

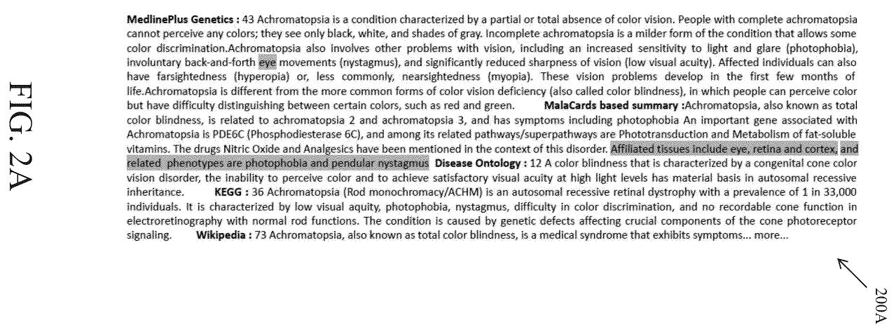

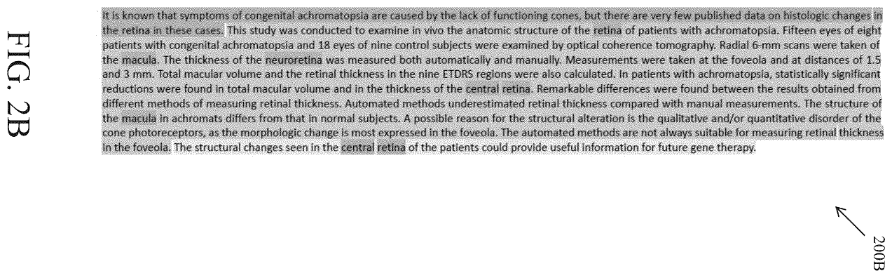

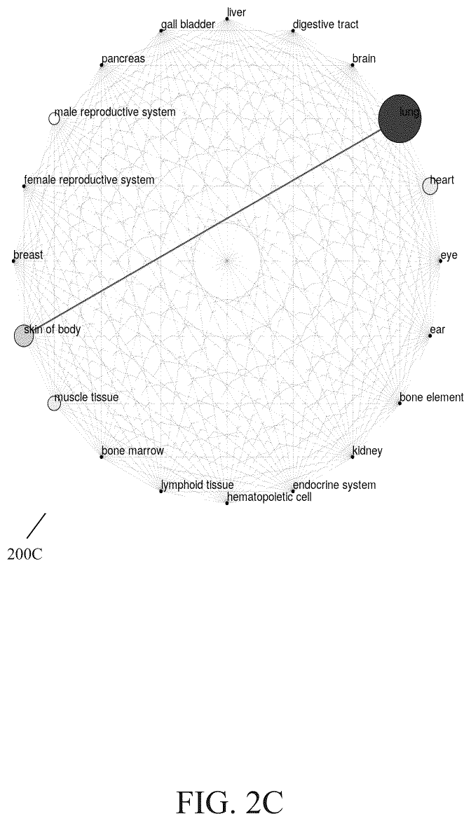

FIG. 1 is a block diagram that illustrates an exemplary system for generating a graph neural network comprising association of one or more organs with a disease. Referring to FIG. 1, a system 100 includes at least one server 108, an organ ontology 102, a plurality of disease datasets 104, a plurality of publication datasets 106. The at least one server 108 comprises an identifier module (not shown), a weight assignor and summation module 110, and a graph neural network generator module 112. The at least one server 108, the organ ontology 102, the plurality of disease datasets 104, the plurality of publication datasets 106 are communicable coupled via a communication network FIG. 1 is described in conjunction FIGS. 2A, 2B and 2C.

The at least one server 108 further comprises a memory, a storage device, an input/output (I/O) device, a user interface, and a wireless transceiver. The organ ontology 102, the plurality of disease datasets 104, the plurality of publication datasets 106 are internal, external or remote resources but communicatively coupled to the at least one server 108 via a communication network.

In some embodiment of the disclosure, the organ ontology 102, the plurality of disease datasets 104, the plurality of publication datasets 106, the identifier module, the weight assignor and summation module 110, and a graph neural network generator module 112 are integrated with other processors and modules to form an integrated system. In some embodiments of the disclosure the one or more processors of the at least one server 108 may be integrated in any order and other combination modules to form an integrated system. In some embodiments of the disclosure, as shown, the organ ontology 102, the plurality of disease datasets 104, the plurality of publication datasets 106, the identifier module, the weight assignor and summation module 110, and a graph neural network generator module 112 and the one or more processors may be distinct from each other. Other separation and/or combination of the various processing engines and entities of the exemplary system 100 illustrated in FIG. 1 may be done without departing from the spirit and scope of the various embodiments of the disclosure.

The identifier module comprises suitable libraries, logic, and/or code that may be operable to implement the identification function in conjunction with the one or more processors. More specifically, the function, in conjunction with the one or more processors, may enable the at least one server 108 to identify one or more organs associated with a disease from one or more categories. In an embodiment, the one or more categories comprises of an organ ontology, one or more information indicative of the disease, plurality of abstract of publications. In an embodiment, the identifier module in conjunction with the organ ontology 102, the plurality of disease datasets 104, the plurality of publication datasets identifies one or more organs associated with a disease from one or more categories. In other embodiment, the identifier module forms an integrated system comprising of the weight assignor and summation module 110, and a graph neural network generator module 112 to generating a graph neural network comprising association of one or more organs with a disease.

In an embodiment, the identifier module receives an input of disease name to associate the disease with one or more organs. The identifier module parses the disease name with an organ ontology 102 to associate the disease with one or more organs. In an embodiment, the disease name includes ‘asbestos-related lung carcinoma’, ‘adrenal cortex disease’, ‘atheroembolism of kidney’, and ‘adult choroid plexus cancer’. In an implementation of the method claimed, the organ identification from the above disease name includes ‘lung’, ‘adrenal cortex’, ‘kidney’, and ‘choroid-plexus’. Based on the normalization of the organ identification it was established that ‘organ-name’-‘lung’ with ‘name-score’-‘1’, ‘organ-name’-‘endocrine system’ with ‘name-score’-1, ‘organ-name’-‘kidney’ with ‘name-score’-‘1’, ‘organ-name’-‘brain’ with ‘name-score’-‘1’, respectively. Therefore, the identifier module based on parsing the disease name with the organ ontology 102 identifies that the diseases ‘asbestos-related lung carcinoma’, ‘adrenal cortex disease’, ‘atheroembolism of kidney’, and ‘adult choroid plexus cancer’ is associated with ‘lung’, ‘endocrine system’, ‘kidney’, ‘brain’ with the corresponding frequency-‘name-score-1’ for each disease.

In an embodiment, the organ ontology 102 comprises organs, tissues, cells and its associated keywords. The organ ontology 102 is created by parsing plurality of organ datasets. In an embodiment, the organ ontology 102 comprises 20 organs and its associated keywords. In an example, the list of organs, tissues and cells is fetched from human protein atlas and uberon ontology database.

In an embodiment, the identifier module parses the one or more information indicative of the disease along with the organ ontology 102 to identify one or more organs associated with the input disease. In an embodiment the one or more information indicative of the disease is extracted from the plurality of disease datasets 104. Referring to FIG. 2A, in an embodiment, the one or more information indicative of the disease 200A is extracted from plurality of disease datasets 104. The identifier module parses the one or more information indicative of the disease 200A to identify the one or more organs associated with the disease, namely, the disease input name is ‘Achromatopsia’, wherein the one or more associated organ is ‘eye’. In an embodiment, the plurality of disease datasets 104 comprises of 18000 disease summary information. In an example, the one or more information indicative of the disease 200A is extracted from 74 different data sources.

In an embodiment, the identifier module parses the plurality of abstract of publications along with the organ ontology 102 to identify one or more organs associated with the input disease. In an embodiment the plurality of abstract of publications is extracted from the plurality of publication datasets 106. Referring to FIG. 2B, in an embodiment, the abstract of publication 200B is extracted from the plurality of publication datasets 106. The identifier module parses the abstract of publication 200B to identify the one or more organs associated with the disease, namely, the disease input name is ‘Achromatopsia’, wherein the one or more associated organ is ‘eye’ and ‘ear’. In an embodiment, parsing the plurality of abstract of publication with the disease name ‘Achromatopsia’, the identifier module identifies ‘eye’, ‘ear’, ‘brain’ and ‘skin of body’ as the potential organs associated with the disease. In an embodiment, the abstract of publication 200B comprises of introduction, methods & materials, and conclusion. The one or more locations are introduction, methods & materials, and conclusion. In an embodiment, the identifier module works in conjunction with other modules to identify the locations in each abstract of the publications.

The weight assignor and summation module 110 comprises suitable libraries, logic, and/or code that may be operable to implement the assignment and summation function in conjunction with the one or more processors. More specifically, the function, in conjunction with the one or more processors, may enable the at least one server 108 to identify one or more organs associated with a disease from one or more categories. In an embodiment, the weight assignor and summation module 110 is operable to assign weights to each of the one or more categories and the identified one or more organs. Further, the weight assignor and summation module 110 is operable to normalize and sum the assigned weights to determine a probability and association indicative of the one or more organ being associated with the disease. The weight assignor and summation module 110 receives data from the identifier module to assign the weights and sum the assigned weights to determine the probability and association indicative of the disease.

Referring to FIG. 1, FIG. 2A and FIG. 2B, in an embodiment, the weight assignor and summation module 110 is operable to assign the following weights based on each of the one or more categories:

| Section | Wt 1 | Wt 2 | Wt 3 | Wt 4 | Wt 5 | Wt 6 | Wt 7 |
|---|---|---|---|---|---|---|---|
| Disease Name | 0.3 | 0.5 | | 0.5 | 0.3 | | |
| Information Indicative | 0.4 | 0.5 | 0.5 | | | 0.4 | |
| of Disease | | | | | | | |
| Abstract of Publication | 0.3 | | 0.5 | 0.5 | | | 0.3 |

Referring to FIG. 2A, the disease input name Achromatopsia the identifier module did not identify any organ from the disease name. The identifier module while working in conjunction with the weight assignor and summation module 110 identifies the organ and the frequency as follows:

| | | Disease | | No. | Sum*No. | |
|---|---|---|---|---|---|---|
| | Organ | Name | | of | of | |
| Organ | Keyword | Dataset | Sum | Source | Source | Normalized |
| eye | eye | 1 | 1 | 1 | 1 | 1 |

Referring to FIG. 2B, the disease input name Achromatopsia the identifier module and the weight assignor and summation module 110 assigns the following weightage to the identified one or more organs and the one or more location of the keywords appearing the plurality of abstracts of publications. The Section L1, L2 and L3 corresponds to the one or more locations, namely, introduction, methods & materials, and conclusion respectively.

Frequency and Weightage Multiplication

| Section | Wt 1 | Wt 2 | Wt 3 | Wt 4 | Wt 5 | Wt 6 | Wt 7 |
|---|---|---|---|---|---|---|---|
| L1 | 0.3 | 0.4 | | 0.5 | 1 | | |
| L2 | 0.4 | 0.6 | 0.6 | | | 1 | |
| L3 | 0.3 | | 0.4 | 0.5 | | | 1 |

| Section | Organ | Freq | Score |
|---|---|---|---|
| L1 | eye | 1 | 0.3 |
| L2 | eye | 3 | 0.3 |
| L2 | eye | 1 | 0.1 |
| L3 | eye | 2 | 0.3 |

In an embodiment, referring to FIG. 2B, the identifier module and the weight assignor and summation module 110 parses the plurality of abstracts from the plurality of publication datasets to identify and determine the following:

Publication collective output

| Organ | Sum | Publication_count | Score | Normalized |
|---|---|---|---|---|
| eye | 68.89 | 223 | 0.31 | 0.65 |
| brain | 29.54 | 223 | 0.13 | 0.28 |
| ear | 2.6 | 223 | 0.01 | 0.02 |
| skin of body | 2.2 | 223 | 0.01 | 0.02 |

In an example, a collective output of the identifier module and the weight assignor and summation module 110 for an input of Achromatopsia is as follows:

Collective Result for Achromatopsia

| Disease Name | Organ | Score |
|---|---|---|
| | — | — |
| Information | Eye | 1 |
| Indicative | | |
| of Disease | | |
| Abstract of | eye | 0.65 |
| Publication | brain | 0.279 |
| | ear | 0.025 |
| | skin of body | 0.021 |

The weights assigned:

| Section | Wt 1 | Wt 2 | Wt 3 | Wt 4 | Wt 5 | Wt 6 | Wt 7 |
|---|---|---|---|---|---|---|---|
| Disease Name | 0.3 | 0.5 | | 0.5 | 0.3 | | |
| Information Indicative | 0.4 | 0.5 | 0.5 | | | 0.4 | |
| of Disease | | | | | | | |
| Abstract of Publication | 0.3 | | 0.5 | 0.5 | | | 0.3 |

The probability of the one or more organ being associated with the disease:

| Organ | Weightage | Score | Normalised probability |
|---|---|---|---|
| eye | (1*.05) + (0.65*0.5) | 0.825 | 0.89 |
| brain | (0.279*0.3) | 0.083 | 0.09 |
| ear | (0.025 * 0.3) | 0.007 | 0.007 |
| skin of body | (0.021*0.3) | 0.006 | 0.006 |

In an embodiment, the weight assignor and summation module 110 is operable to determine the association indicative of the one or more organ being associated with the disease from the plurality of abstract of publications. In an example, the module 110 will provide the following out for every publication, wherein list of organs are the associations indicated in the one or more organs being associated with the disease.

| Publication | Organ |
|---|---|
| 1 | Lung |
| 2 | Lung |
| 3 | Lung |
| 4 | Lung |
| 5 | heart, digestive tact |
| 6 | heart, lung |
| 7 | bone element, bone marrow, lung |
| 8 | digestive tact, lung, liver |
| 9 | digestive tact, lung, |

The graph neural network generator module 112 comprises libraries, logic, and/or code that may be operable to generating a graph neural network comprising association of one or more organs with a disease. In an embodiment, the graph neural network is generated based on the probability and the association indicative of the one or more organ being associated with the disease. The identifier module and the weight assignor and summation module 110 provides probability of the one or more organ being associated with the disease and association indicative of the one or more organ. of the one or more organ being associated with the disease. Probability of the one or more organ being associated with the disease denotes the probability of the said organ being associated with the respective disease; while the association indicative specifies the combination of the organs coming from every publication. The probability and the association indicative of the one or more organ being associated with the disease is further used for generation of a graph network by the graph neural network generator module 112. Referring to FIG. 2C, it denotes a graph neural network generated for a disease. The node size of the FIG. 2C is denoted by the probability of the organ being associated with the disease; while the edges are denoted by the combination presence in the association indicative of the one or more organ. Color intensity (red to white) and node size represents the probability of the organ being associated with the disease. Connections between the organs are shown in the Blue line. Black nodes are the organs present in the ontology but having no association with the disease; and the gray lines are the underlying connection but not present in the organ search output data

The database may be capable of providing mass storage to the at least one server 108. In some embodiments, the database may be or contain a computer-readable medium, such as a hard disk device, an optical disk device, or a tape device, a flash memory or other similar solid state memory device, or an array of devices, including devices in a storage area network or other configurations. A computer program product may be tangibly embodied in an information carrier. The information carrier may be a computer-readable or machine-readable medium, such as database. The computer program product may also contain instructions that, when executed, perform one or more methods, such as those described in the disclosure.

A user interface (not shown) may comprise suitable logic, circuitry, and interfaces that may be configured to present the results i.e., the generated graph neural network. In an embodiment, the user interface would receive the disease name input to initiate the method for generating a graph neural network comprising association of one or more organs with a disease. The results are presented in form of an audible, visual, tactile, or other output to the user, such as a researcher, a scientist, a principal investigator, data manager, and a health authority, associated with the at least one server 108. As such, the user interface may include, for example, a display, one or more switches, buttons, or keys (e.g., a keyboard or other function buttons), a mouse, and/or other input/output mechanisms. In an example embodiment, the user interface may include a plurality of lights, a display, a speaker, a microphone, and/or the like. In some embodiments, the user interface may also provide interface mechanisms that are generated on the display for facilitating user interaction. Thus, for example, the user interface may be configured to provide interface consoles, web pages, web portals, drop down menus, buttons, and/or the like, and components thereof to facilitate user interaction.

The communication network may be any kind of network, or a combination of various networks, and it is shown illustrating exemplary communication that may occur between the plurality of data sources 102, 104 and 106, and the at least one server 108. For example, the communication network may comprise one or more of a cable television network, the Internet, a satellite communication network, or a group of interconnected networks (for example, Wide Area Networks or WANs), such as the World Wide Web. Although one mode of communication network the communication network is shown, the disclosure is not limited in this regard. Accordingly, other exemplary modes may comprise uni-directional or bi-directional distribution, such as packet-radio, and satellite networks.

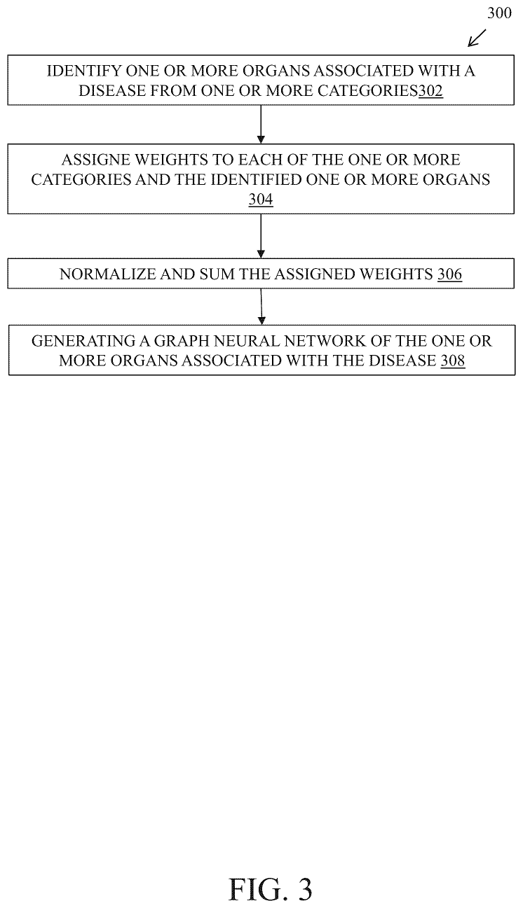

FIG. 3 depict flowcharts illustrating exemplary operations for generating a graph neural network comprising association of one or more organs with a disease. Flowchart 300 is described in conjunction with FIG. 1.

At step 302, one or more organs associated with a disease is identified from one or more categories. The categories comprises of an organ ontology, one or more information indicative of the disease, plurality of abstract of publications.

At step 304, each of the one or more categories and the identified one or more organs are assigned weights. The weights are assigned based on one or more of—frequency of the keywords appearing the one or more categories, and one or more location of the keywords appearing the plurality of abstracts of publications.

At step 306, determine a probability and association indicative of the one or more organ being associated with the disease. The probability and the association indicative of the one or more organ being associated with the disease is determined by the weight assignor and summation module 110.

At step 308, generate a graph neural network of the one or more organs associated with the disease. The graph neural network is based on the probability and the association indicative of the one or more organ being associated with the disease.

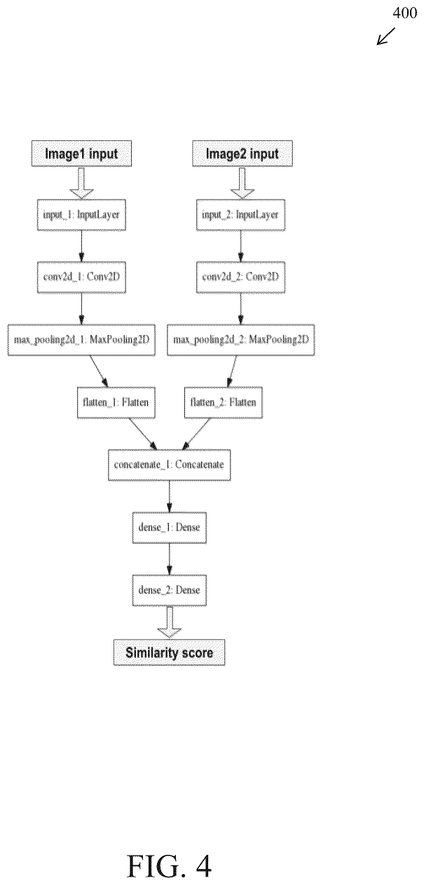

FIG. 4 illustrates an example of a CNN Model architecture for comparing the graph neural network of the one or more organs associated with one or more diseases, in accordance with an exemplary embodiment of the disclosure. Referring to FIG. 4, the model CNN architecture 400 is configured to similar diseases of one or more organs, as described herein.

The model CNN architecture 400 is operable to receive the generated graph neural network for one or more organs associated with a disease. In an embodiment, for generating the similarity score of the one or more associated with one or more diseases, one or more graph neural network is generated for each of the input disease to identify the one or more organs associated with each disease of the one or more diseases.

In an embodiment, the input image 1 and input image 2 corresponds to the generated one or more graph neural networks. An input layer associated with the CNN model architecture 400 to generate a Con2D image of the one or more graph neural networks. In an embodiment, a pooling module is operable to receive the Con2D image and maxpool2d the one or more graph neural networks.

In an embodiment, the one or more graph neural networks are fatten from the maxpool2d module to further concatenate and dense the images to generate the similarity score.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and/or code (if any is necessary) to perform the function, regardless of whether performance of the function is disabled, or not enabled, by some user-configurable setting.

The terminology used herein is for the purpose of describing particular embodiments only and is not intended to be limiting of embodiments of the disclosure. As used herein, the singular forms “a”, “an” and “the” are intended to include the plural forms as well, unless the context clearly indicates otherwise. It will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any non-transitory form of computer readable storage medium having stored therein a corresponding set of computer instructions that upon execution would cause an associated processor to perform the functionality described herein. Thus, the various aspects of the disclosure may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

Another embodiment of the disclosure may provide a non-transitory machine and/or computer-readable storage and/or media, having stored thereon, a machine code and/or a computer program having at least one code section executable by a machine and/or a computer, thereby causing the machine and/or computer to perform the steps as described herein for determining combination drug and use in pancreatic cancer treatment.

The present disclosure may also be embedded in a computer program product, which comprises all the features enabling the implementation of the methods described herein, and which when loaded in a computer system is able to carry out these methods. Computer program in the present context means any expression, in any language, code or notation, either statically or dynamically defined, of a set of instructions intended to cause a system having an information processing capability to perform a particular function either directly or after either or both of the following: a) conversion to another language, code or notation; b) reproduction in a different material form.

Further, those of skill in the art will appreciate that the various illustrative logical blocks, modules, circuits, algorithms, and/or steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The methods, sequences and/or algorithms described in connection with the embodiments disclosed herein may be embodied directly in firmware, hardware, in a software module executed by a processor, or in a combination thereof. A software module may reside in RAM memory, flash memory, ROM memory, EPROM memory, EEPROM memory, registers, hard disk, physical and/or virtual disk, a removable disk, a CD-ROM, virtualized system or device such as a virtual server or container, or any other form of storage medium known in the art. An exemplary storage medium is communicatively coupled to the processor (including logic/code executing in the processor) such that the processor can read information from, and write information to, the storage medium. In the alternative, the storage medium may be integral to the processor.

While the present disclosure has been described with reference to certain embodiments, it will be noted understood by, for example, those skilled in the art that various changes and modifications could be made and equivalents may be substituted without departing from the scope of the present disclosure as defined, for example, in the appended claims. In addition, many modifications may be made to adapt a particular situation or material to the teachings of the present disclosure without departing from its scope. The functions, steps and/or actions of the method claims in accordance with the embodiments of the disclosure described herein need not be performed in any particular order. Furthermore, although elements of the disclosure may be described or claimed in the singular, the plural is contemplated unless limitation to the singular is explicitly stated. Therefore, it is intended that the present disclosure is not limited to the particular embodiment disclosed, but that the present disclosure will include all embodiments falling within the scope of the appended claims.

What is claimed is:

1. A method for generating a graph neural network comprising association of one or more organs with a disease, wherein the method comprises
Identifying one or more organs associated with a disease from one or more categories, wherein the one or more categories comprises of an organ ontology, one or more information indicative of the disease, plurality of abstract of publications,
Assigning weights to each of the one or more categories and the identified one or more organs, wherein the weights are assigned to the identified one or more organs based on one or more of—frequency of the keywords appearing the one or more categories, and one or more location of the keywords appearing the plurality of abstracts of publications,
Normalizing and summing the assigned weights to determine a probability and association indicative of the one or more organ being associated with the disease,
Generating a graph neural network of the one or more organs associated with the disease, wherein the graph neural network is based on the probability and the association indicative of the one or more organ being associated with the disease.

2. The method as claimed in claim 1, comprises generating disease similarity score for one or more diseases based on the graph neural network for one or more organ associated with one or more disease.

3. The method as claimed in claim 2, wherein the disease similarity score is generated based on implementing a CNN Model architecture for comparing the graph neural network of the one or more organs associated with one or more diseases.

4. The method as claimed in claim 1, comprises creating, by one or more processors, the organ ontology, wherein the organ ontology comprises organs, tissues, cells and its associated keywords.

5. The method as claimed in claim 4, wherein creating the organ ontology comprises parsing plurality of organ datasets.

6. The method as claimed in claim 1, wherein the method comprises extracting information indicative of the disease from a plurality of disease datasets.

7. The method as claimed in claim 1, wherein the method comprises extracting plurality of abstract from a plurality of publication datasets.

8. The method as claimed in claim 1, wherein the one or more locations in the abstract of the publication comprising introduction, methods & materials, and conclusion.

9. The method as claimed in claim 1, wherein a higher weightage is assigned to the identified one or more organs for higher frequency of the keywords appearing in—the one or more information indicative of the disease, and methods & materials location of the one or more location in the abstract.

10. The method as claimed in claim 1, wherein the probability output denotes a probability of the said organ being associated with the respective disease, and the association indicative of the one or more organ being associated with the disease specifies a combination of the one or more organs extracted from each of the abstract of the publication.

11. A system for generating a graph neural network comprising association of one or more organs with a disease, comprising of
an organ ontology comprising of organs, tissues, cells and its associated keywords;
a plurality of disease datasets comprising of information indicative of the disease;
a plurality of publication datasets comprising of plurality of abstract of publications;
at least one server communicably coupled with the organ ontology, the plurality of disease datasets, and the plurality of publication datasets, comprising one or more processors configured to:
Identify one or more organs associated with a disease from one or more categories, wherein the one or more categories comprises of an organ ontology, one or more information indicative of the disease, plurality of abstract of publications,
Assign weights to each of the one or more categories and the identified one or more organs, wherein the weights are assigned to the identified one or more organs based on one or more of—frequency of the keywords appearing the one or more categories, and one or more location of the keywords appearing the plurality of abstracts of publications,
Normalize and sum the assigned weights to determine a probability and association indicative of the one or more organ being associated with the disease, and
Generate a graph neural network of the one or more organs associated with the disease, wherein the graph neural network is based on the probability and the association indicative of the one or more organ being associated with the disease.

12. The system as claimed in claim 11, wherein the at least one server is configured to generate disease similarity score for one or more diseases based on the graph neural network for one or more organ associated with one or more disease.

13. The system as claimed in claim 12, the disease similarity score is generated based on implementing a CNN Model architecture for comparing the graph neural network of the one or more organs associated with one or more diseases.

14. The system as claimed in claim 11, wherein the organ ontology is created by parsing plurality of organ datasets

15. The system as claimed in claim 11, wherein the one or more locations in the abstract of the publication comprising introduction, methods & materials, and conclusion.

16. The system as claimed in claim 11, wherein a higher weightage is assigned to the identified one or more organs for higher frequency of the keywords appearing in—the one or more information indicative of the disease, and methods & materials location of the one or more location in the abstract.

17. The system as claimed in claim 11, the probability output denotes a probability of the said organ being associated with the respective disease, and the association indicative of the one or more organ being associated with the disease specifies a combination of the one or more organs extracted from each of the abstract of the publication.

18. A computer program product comprising a computer useable medium having computer program logic recorded thereon for enabling a processor to generate a graph neural network comprising association of one or more organs with a disease, the computer program logic comprising:
Identify one or more organs associated with a disease from one or more categories, wherein the one or more categories comprises of an organ ontology, one or more information indicative of the disease, plurality of abstract of publications,
Assign weights to each of the one or more categories and the identified one or more organs, wherein the weights are assigned to the identified one or more organs based on one or more of—frequency of the keywords appearing the one or more categories, and one or more location of the keywords appearing the plurality of abstracts of publications,
Normalize and sum the assigned weights to determine a probability and association indicative of the one or more organ being associated with the disease, and
Generate a graph neural network of the one or more organs associated with the disease, wherein the graph neural network is based on the probability and the association indicative of the one or more organ being associated with the disease.
